SEQUENCE LISTING (1 sequence)

SEQ ID NO: 1 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

(19) United States
(12) Patent Application Publication
(10) Pub. No.: US 2025/0213682 A1
(43) Pub. Date: Jul. 3, 2025

(54) Modulation of Interleukin 17F for Treatment of Infection and Organ Dysfunction

(71) Applicant: THE FEINSTEIN INSTITUTES FOR MEDICAL RESEARCH, Manhasset, NY (US)

(72) Inventor: Matthew D. Taylor, Manhasset, NY (US)

(73) Assignee: THE FEINSTEIN INSTITUTES FOR MEDICAL RESEARCH, Manhasset, NY (US)

(21) Appl. No.: 18/983,954

(22) Filed: Dec. 17, 2024

Related U.S. Application Data
(60) Provisional application No. 63/615,002, filed on Dec. 27, 2023.

(51) Int. Cl. A61K 39/395; A61K 39/00; A61P 37/04

(52) U.S. Cl. CPC A61K 39/3955; A61P 37/04; A61K 2039/505; A61K 2039/552; A61K 2039/58

(57) ABSTRACT

Provided are methods of treating an infection or an organ dysfunction in a patient with a disease or disorder comprising modulating the level or activity of interleukin (IL)17F in the patient. In one embodiment, the patient has an infection and supplementation of IL17F augments clearance of the infection in the patient. In another embodiment. blockade of IL17F ameliorates organ dysfunction in the patient.

### CROSS-REFERENCE TO RELATED APPLICATIONS

This application claims the benefit of U.S. Provisional Patent Application No. 63/615,002, filed on Dec. 27, 2023, the contents of which are herein incorporated by reference in their entirety into the present application.

### STATEMENT OF GOVERNMENT SUPPORT

This invention was made with government support under grant no. GM132794 awarded by the National Institutes of Health. The government has certain rights in the invention.

### INCORPORATION BY REFERENCE OF SEQUENCE LISTING

The Sequence Listing entitled “50425-830.xml”, 2 KB, created on Nov. 18, 2024 and submitted herewith electronically using the U.S. Patent Center is incorporated by reference as the Sequence Listing XML for the subject application.

### BACKGROUND

Throughout this application various publications are referred to in parentheses. Full citations for these may be found at the end of the specification immediately preceding the claims. The disclosures of these publications are hereby incorporated by reference in their entireties into the subject application to more fully describe the art to which the subject application pertains. The discussion of these publications herein is intended merely to summarize the assertions made by Applicant and no admission is made that any publication constitutes prior art.

Sepsis is defined as life-threatening organ dysfunction caused by a dysregulated host response to infection (1). A majority of studies using murine models of sepsis have been performed in mice maintained in specific pathogen free environments, preventing development of a memory T cell compartment and altering sepsis responses (2-4). We have developed a method to enhance understanding of the effects of memory CD4 and CD8 T cell cytokine production on sepsis-associated immune responses and organ dysfunction utilizing the cecal ligation and puncture (CLP) model of sepsis. This method increases the fidelity of CLP to human sepsis with more similar T cell responses and increased organ dysfunction. We have termed this improved model the Immune Educated CLP model. To develop Immune Educated animals, we intravenously administered an anti-CD3 activating antibody 30 days prior to CLP. This treatment induced combined CD4 and CD8 T cell memory without specificity to allow the study of non-specific T cell memory in a model without a specific infectious agent. The T cell compartment of this model more closely resembles the human T cell repertoire and therefore Immune Educated CLP more closely models human sepsis (13).

Our work in Immune Educated CLP, and other work in different models, has identified that T cell memory contributes significantly to the dysregulated host response that is a defining characteristic of sepsis. Our data indicate that a deficiency of memory T cells may have limited translation of findings from mouse models to human sepsis (5-7). Indeed, we recently demonstrated that T cell memory alters both innate immune responses and organ dysfunction in the murine cecal ligation and puncture (CLP) model of sepsis. Specifically, combined polyclonal CD4 and CD8 T cell memory led to more pronounced hepatic neutrophilic infiltration, enhanced monocyte and macrophage inflammatory responses and increased hepatic dysfunction following CLP. Individually, CD4 T cell memory or CD8 T cell memory did not recapitulate these findings. Increases in hepatic neutrophilic infiltration were only found in the presence of both CD4 and CD8 T cell memory. These findings indicated that memory induction endowed each of these cell lineages with specific and non-redundant effector functions which contributed to the sepsis immunopathological response for which the opposing lineage failed to compensate (6).

The major acute cause of morbidity in sepsis is the development of multi-system organ dysfunction, leading to subsequent organ failure and mortality, both of which are enhanced by T cell memory associated immune responses (6, 8, 9). Hepatic dysfunction occurs in up to 35% of sepsis patients and is associated with particularly poor outcomes, with up to 55% mortality in patients with sepsis-associated hepatobiliary dysfunction (10, 11), The liver maintains hematologic stability through production of coagulation factors and elimination of wastes including bile acids and bilirubin. This organ also acts as a first line of immunologic defense by clearing bacteria and other inflammatory products from both the gut and the systemic circulation. Hepatic neutrophils, monocytes and macrophages are essential to these immune functions, but the effects of T cell responses on the liver, while heavily studied in classical idiopathic liver failure and immune activation cytokine storm syndromes like macrophage activation syndrome and hemophagocytic lymphohistiocytosis, have not been well examined in sepsis (12). Further, the effects of T cell memory responses on sepsis-associated hepatic dysfunction are not well understood. Memory T cells gain the ability to produce a far greater range of cytokines compared to naïve T cells; cytokine production by activated memory T cells may contribute to altered immune responses and hepatic function in CLP and sepsis.

Many studies have investigated interleukin 17 (IL17) as a cytokine produced primarily by T cells that is important in modulating immunity to bacterial, mycobacterial and fungal pathogens. IL17 has many isoforms (IL17A-IL17F), with IL17A being the most studied. T cells are the major source of IL-17 production and are able to synthesize both the IL17A and IL17F isoforms of the cytokine. In inflammatory colitis, IL17F has been shown to drive IFNg+ T cell responses (57). IL17A has been widely shown to drive neutrophil and monocyte inflammatory responses but these have not been directly attributed to IL17F. IL17F has approximately 50% structural homology and overlap in amino acid sequence identity with IL17A. The structure of IL17F is known and described (55, 56). Further, both IL17A and IL17F act on the same receptors (IL17RA and IL17RC), though there is differential affinity for these receptors—IL17A primarily binds to IL17RA, and IL17F binds preferentially to IL17RC.

Sepsis is a fulminant acute syndrome caused by a dysregulated response to infection. There is evidence that IL17F plays a role in less fulminant infection and can regulate immune responses in both normal infection and in autoimmunity. There is evidence that IL17F plays a role in inflammatory bowel disease or rheumatoid arthritis, though there is overlap in these disease processes with IL17A and the individual roles are not completely clear (57,58).

The present invention uses modulation of IL17F to address the need for treatments to reduce organ dysfunction and promote clearance of infection in diseases and disorders such as sepsis.

### SUMMARY OF THE INVENTION

The invention provides a method of treating an infection or an organ dysfunction in a patient with a disease or disorder comprising modulating the level or activity of interleukin (IL)17F in the patient. In one embodiment, the patient has an infection and supplementation of IL17F augments clearance of the infection in the patient. In another embodiment. blockade of IL17F ameliorates organ dysfunction in a patient with a disease or disorder.

These and other embodiments are disclosed or are obvious from and encompassed by the following Detailed Description of the Invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1A-1C. Effects of Immune Education and Specific Cytokine Blockade on the Serum Cytokine Response to CLP. Immune Educated or Control C57Bl/6 mice were sacrificed 24 hrs. following CLP and serum levels of (A) IFNγ, (B) IL12, (C) IL17 (non-specific) were assessed. Data as mean±SD. Analyzed by 2-way ANOVA with Tukey's Test for Multiple Comparisons. *=p<0.05 for comparisons between two groups, #=p<0.05 for an effect of cytokine blockade on the CLP response compared to untreated CLP. Majority of graphs shown on logarithmic scale and log₁₀ transformation used prior to statistical analysis for skewed variability. N=4-13/group. Columns from left to right in each pair represent, respectively, Control and Educated.

FIG. 2A-2B. Effects of Immune Education, CLP, and Specific Cytokine Blockade on T cell Cytokine Production. (A-B) Immune Educated or Control C57Bl/6-Il17a^{tm1Begen}Il17f^{em1Litt}/J IL17A^{EGFP}/IL17F^{mcherry} reporter mice were sacrificed prior to or 24 hrs. following CLP. IL17A⁺ and IL17F⁺ T cells were assessed. Percent IL17A⁺ CD4 (Left), IL17A⁺ CD8 (Middle Left), IL17F⁺ CD4 (Middle Right) and IL17F⁺ CD8 (Right) T cells in the (A) inguinal lymph node (ILN) and (B) liver.

FIG. 3A-3C. Effects of Immune Education, CLP, and Specific Cytokine Blockade on CLP-Induced Hepatic Dysfunction. Immune Educated or Control C57Bl/6 mice were sacrificed prior to or 24 hrs. following CLP alone or with specific cytokine blockade. (A) Hepatic tissue was assessed by RT-PCR for log₁₀ transformed relative transcription levels of SCLO1a1, SLC10, and ABCC2. (B) Serum Alanine aminotransferase. (C) Log₁₀ transformed peritoneal bacterial colony forming units. Data as mean±SD. Analyzed by 2-way ANOVA with Sidak's Test for Multiple Comparisons. *=p<0.05 for comparisons between two groups. IL17A and IL17F blockade compared to each other separately from other comparisons. Log₁₀ transformation used prior to statistical analysis for skewed variables. N=4-22/group. Columns from left to right in each pair represent, respectively, Control and Educated.

### DETAILED DESCRIPTION OF THE INVENTION

The invention provides methods of treating an infection or an organ dysfunction in a patient with a disease or disorder comprising modulating the level or activity of interleukin (IL)17F in the patient.

In one embodiment, the patient has an infection and supplementation of IL17F augments clearance of the infection in the patient. The infection can be, for example, a bacterial, mycobacterial, fungal or viral infection. The infection can be due, for example, to influenza, Epstein-Barr virus (EBV), Cytomegalovirus (CMV), a hemorrhagic fever virus or COVID19. The infection can be, e.g., a persistent chronic infection or a severe acute infection. In one embodiment, the patient has sepsis and supplementation of IL17F augments bacterial clearance in the patient. In one embodiment, the patient has pneumonia or hepatitis or encephalitis.

In one embodiment, blockade of IL17F ameliorates organ dysfunction in the patient with the disease or disorder. The organ dysfunction can be, for example, one or more of hepatic, renal and lung dysfunction. The disease or disorder can be, for example, severe trauma, a severe burn, brain injury/stroke, lung injury/acute respiratory distress syndrome (ARDS), drug overdose, acute renal failure (ARF), idiopathic liver failure, virally induced liver failure, hemophagocytic lymphohistiocytosis (HLH), macrophage activation syndrome (MAS), systemic Juvenile Idiopathic Arthritis (sJIA), systemic lupus erythematosus (SLE) or Kawasaki's disease (KD). Blocking IL17F should also play a role in ameliorating organ dysfunction in other syndromes of critical illness associated with significant organ dysfunction in the setting of a systemic inflammatory response. In one embodiment, the patient has sepsis and blockade of IL17F ameliorates hepatic dysfunction.

Treatment for a patient with sepsis will depend on the clinical situation. A subset of sepsis patients have persistent bacterial infection. These patients will benefit from supplementation of IL17F to boost bacterial clearance with a possible risk of increased organ dysfunction as a side effect. Another subset of sepsis patients may have significant persistent organ dysfunction. These patients will benefit from blockade of IL17F, especially if a bacterial infection has already been controlled with antibiotics. In sum, certain patients will benefit from IL17F supplementation, while other patients will benefit from IL17F blockade.

Supplementation of IL17F can be done, for example, through infusion of purified or recombinant IL17F. Recombinant human IL17 is available from BioLegend, SanDiego, California, Catalog #570606 (biolegend.com/en-us/products/recombinant-human-il-17f-carrier-free-6400).

Blockade of IL17F can be performed, for example, through antibody therapy to either IL17F or to its primary receptor IL17RC. A monoclonal antibody to IL17F is available from BioCell, Lebanon, New Hampshire, Catalog #BE0303 (bioxcell.com/invivomab-anti-mouse-il-17f-be0303).

Human IL17 has the following amino acid sequence (Ensembl.org, grch37.ensembl.org/Homo_sapiens/Transcript/Sequence_Protein?db=core;g=ENSG0000011 2116;r=6:52101479-52109335;t=ENST00000336123) (SEQ ID NO:1):

MTVKTLHGPAMVKYLLLSILGLAFLSEAAARKIPKVGHTFFQKPESCPPV
PGGSMKLDIGIINENQRVSMSRNIESRSTSPWNYTVTWDPNRYPSEVVQA
QCRNLGCINAQGKEDISMNSVPIQQETLVVRRKHQGCSVSFQLEKVLVTV
GCTCVTPVIHHVQ.

The nucleic acid sequence for Human IL17F is known (Ensembl.org, grch37.ensembl.org/Homo_sapiens/Transcript/Sequence_cDNA?db=core;g=ENSG00000112 116;r=6:52101479-52109335;t=ENST00000336123).

Bacterial clearance in a patient can be assessed through several ways, including, for example:
A. negative serial blood cultures,
B. reduction in markers of bacterial infection—e.g., C-reactive protein (CRP), procalcitonin (PCT), white blood cell count (WBC), absolute neutrophil count (ANC), and/or neutrophil to lymphocyte ratio (NLR), and/or
C. improvement in clinical condition.

Hepatic dysfunction similarly can be assessed through several measures, including. For example:
A. elevated hepatic markers of injury—e.g., increased alanine aminotransferase (ALT), aspartate aminotransferase (AST), alkaline phosphatase (ALP), gamma-glutamyl transferase (GGT), lactate dehydrogenase (LDH), and/or ferritin,
B. elevated markers of liver clearance—e.g., bilirubin levels, and/or ammonia levels, and/or
C. altered markers of liver synthesis—e.g., increased coagulation measurements (e.g., Prothrombin time/PT, Partial thromboplastin time/PTT, international normalized ratio/INR), decreased serum total protein, and/or decreased serum albumin).

In one embodiment, treatment of the patient does not comprise administering a substance to the patient that directly modulates the level or activity of IL17A.

The patient can be a human or a veterinary animal.

### Experimental Details

### Overview

In this work, we set out to understand the effects of memory CD4 and CD8 T cell cytokine production on sepsis-associated immune responses and organ dysfunction utilizing the CLP model. In a subset of animals, we used an anti-CD3 activating antibody, administered intravenously to animals 30 days prior to CLP, to induce combined CD4 and CD8 T cell memory without specificity to allow the study of non-specific T cell memory in a model without a specific infectious agent.(13) Indeed, we found that naïve CD8 T cells in both the liver and the inguinal lymph nodes produce IL17F following CLP, contributing to the development of monocyte-derived dendritic cells (MoDCs). These cells in turn produce IL12, which both promotes further hepatic MoDC development and suppresses CD4 and CD8 T cell TNFα production and CD4 IL17A responses, while promoting IFNγ production by CD8 T cells. IFNγ in turn causes significant augmentation of the hepatic MoDC response, suppresses hepatic neutrophil accumulation while augmenting hepatic neutrophil cytokine production, and causes hepatic dysfunction in the CLP model of sepsis. This same response, mediated by IL17F and IFNγ assist in bacterial clearance in this model, and blockade of these cytokines, reduces immune mediated bacterial clearance. Further, peripheral cytokine analysis in human sepsis patients demonstrates that those with hepatic dysfunction demonstrates have higher levels of several IFNγ responsive cytokines when compared to those without hepatic dysfunction.

### Methods

### Study Approval

All animal studies were approved by the Institutional Animal Care and Use Committee (IACUC #2017-039) and adhered to National Institutes of Health and Animal Research: Reporting of In Vivo Experiments (ARRIVE) guidelines.

### Mice

C57Bl/6J male mice were obtained from the Jackson Laboratory (Bar Harbor, ME) and maintained in the animal facility at the Feinstein Institutes for Medical Research. CD4^{-/-} (B6.129S2-CD4^{tm1Mak}/J), CD8^{-/-} (B6.129S2-CD8a^{tm1Mnk}/J), IFNγ^{-/-} (B6.129S7-Ifng^{tm1Ts}/J IFNγ), IFNγ-eYFP reporter mice (B6.129S4-Ifng^{tm3.1Lky}/J), and IL17A-eGFP/IL17F-mCherry reporter mice (C57Bl/6-Il17^{atm1Bcgen}Il17^{fem1Litt}/J IL17A^{EGFP}/IL17Fm^{Cherry}) were obtained from the Jackson Laboratory and bred and maintained in our immunodeficient animal facility.

### Cecal Ligation and Puncture Procedure

CLP was performed by an experienced surgeon with two 22-gauge punctures under isoflurane anesthesia as previously described.(5, 6, 48, 49) Sham operation was performed in an identical fashion to CLP, but without ligation and puncture. All animals that underwent CLP were approximately 12 weeks of age. Animals were resuscitated with 50 mL/kg sterile normal saline at the end of surgery through subcutaneous tissue injection. This was repeated every 24 hours for up to 48 hours. This procedure produces approximately a 50% mortality at 48 hrs. Mice were euthanized at 24 hours after CLP with terminal bleed followed by cervical dislocation.

### In Vivo Immunization

Ultra-LEAF Anti-mouse CD3P Antibody (145-2C11) was obtained from Biolegend along with Ultra-LEAF isotype Armenian Hamster IgG control (HTK888). Antibodies were used for T cell activation in vivo and memory T cell development as previously described.(13) 50 μg antibody were given in 200 μL sterile phosphate-buffered saline through retro-orbital venous sinus injection at approximately 7 weeks of age. Mice were monitored for adverse effects and rested for 35 days to allow for memory development and to avoid any continued activation effects.

### In Vivo Cytokine Blockade

Mice were administered monoclonal antibody blockade at the time of inflammatory challenge and every three days following challenge. IFNγ (XMG1.2, 0.5 mg), IL12p40 (C17.8, 1 mg), TNFα (XT3.11, 1 mg), IL17A (17F3, 0.5 mg) and IL17F (MM17F8F5.1A9, 0.5 mg) blocking antibodies were all obtained from BioXCell (Lebanon, NH), diluted from stock concentration in sterile PBS and administered through intraperitoneal injection. Full blocking doses as described in previous literature were used to identify cytokine effects.(50-53)

### Adoptive Transfer

C57Bl/6J or IFNγ^{-/-} (B6.129S7-Ifng^{tm1Ts}/J IFNγ) mice were sacrificed, spleens were homogenized and T cells were isolated by negative selection using either the CD4 or CD8 T cell isolation kits (Miltenyi). 10 million T cells were injected into CD4^{-/-} (B6.129S2-CD4^{tm1Mak}/J, received CD4 T cells) or CD8^{-/-} (B6.129S2-CD8a^{tm1Mak}/J, received CD8 T cells) mice. Controls were given saline vehicle. Thirty days later, mice underwent CLP and were sacrificed for analysis at 24 hrs.

Organ Weights and Leukocyte Isolation Spleens, lymph nodes and livers were obtained from sacrificed mice and immediately weighed. Spleen cells were obtained from spleens digested with DNAse (100 μg/mL) and Collagenase A (1 mg/mL) in complete media for 30 minutes at 37° C. and resuspended following filtration through a 70 μm filter. Red blood cells were lysed, cells were counted using a Countess II Automated Cell Counter (ThermoFisher) and spleen cells were analyzed. Liver leukocytes were obtained following homogenization of livers and filtration through a 70 μm filter. Leukocytes were isolated at the interface of a 35%/75% Percoll solution gradient (GE Healthcare). Inguinal lymph node cells were obtained following homogenization of lymph nodes and filtration through a 70 μm filter. Cells were counted using a Countess II Automated Cell Counter.

### Flow Cytometric Analysis

Once single-cell suspensions were obtained, cells were stained for flow cytometric analysis. Staining was performed with LIVE/DEAD fixable viability dye (Life Technologies) and the following antibodies: CD90.2, CD44, CD8a, CD4, CD62L, CD11a, Ly6C, CD11c, Ly6G, B220, NK1.1, MHCII, IL1b, TNFa, IL2, IFNg and IL17A. All flow cytometric analysis was performed on a BD LSR Fortessa 16-color cell analyzer and analyzed using FlowJo software version 10. A minimum of 2×10⁶ events were analyzed for each sample.

Once single-cell suspensions were obtained, cells were stained for flow cytometric analysis. Staining was performed with LIVE/DEAD fixable viability dye (Life Technologies) and the following antibodies: CD90.2, CD8a, CD4, CD69, Ki67, PD1, Ly6C, CD11c, Ly6G, MHCII, MHCI, CD86, CD80, CD40, IFNγ, IL12p40, and Granzyme B. All flow cytometric analysis was performed on a BD LSR Fortessa 16-color cell analyzer and analyzed using FlowJo software version 10 (BD Bioscience, San Jose, CA). Gating strategies are listed in figure captions.

### Cytokine Production Assays

To assess cytokine production, once cells were in single cell suspension, they were placed with appropriate stimuli. For T cells, T cell receptor stimulation was performed through 20 plate bound anti-CD3e (5 ug/ml) and anti-CD28 (1.7 ug/ml) in solution and for PMA/Ionomycin stimulation PMA (50 ng/ml) and ionomycin (1.5 uM) were used. Cells were stimulated for 5 hours in the presence of Brefeldin A (2 ug/ml). For innate cells, cells were stimulated with LPS (500 ng/ml) for 3 hours in the presence of Brefeldin A. All stimulation assays were performed alongside a control without stimulation to assess for background production as previously described.(51)

### ELISA Assays

Multiplex ELISA assays were performed by EVE Technologies (Calgary, AB, Canada). Findings were confirmed with BD OptEIA ELISA kits. Alanine Aminotransferase 1 ELISA Kit was used per manufacturer instructions (Biovision).

### Real-Time Polymerase Chain Reaction

Samples were stored in RNALater solution (Invitrogen) until total RNA was isolated (RNAeasy Mini Kit; Qiagen). Quantitative real-time PCR was performed using an Applied Biosystems 7900HT Fast Real-Time PCR system. Primers were obtained from Applied Biosystems: TaqMan Gene Expression Assays Mm00496899_m1 (ABCC2), Mm00441421_m1 (SLC10a1), Mm01267415_m1 (SLCO1a1), and Mm99999915_g1 (Gapdh). Relative amplification was calculated using the ΔΔCT method.

### Bacterial Cultures

Peritoneal wash fluid was diluted serially and plated on trypticase soy agar with 5% sheep blood plates. Colonies were counted after 24 hours incubation at 37° C.

### Statistical Analysis

Animal data were analyzed using Student's two-way T test or using two-way analysis of variance with Sidak's Test for Multiple Comparisons where appropriate (Prism 7.0; GraphPad). All data shown are representative of at least two independent experiments to ensure results.

### Results

### Differential Memory Versus Nave T Cell Cytokine Responses Following Cecal Ligation and Puncture

Naïve T cells typically must undergo an initial activation phase during the primary response to antigen to gain effector functions like cytokine production or proliferation.(14) Memory T cells can mount effector responses in a much more acute timeframe. (15, 16) Our laboratory and others have previously demonstrated increased serum cytokine responses to the cecal ligation and puncture (CLP) model of sepsis in the presence of induced T cell memory prior to challenge,(6, 7) but the role of memory T cell-derived cytokine production following CLP has not been thoroughly assessed. Following T cell receptor (TCR) stimulation of cells obtained from standard laboratory mice without prior T cell memory induction, a greater percentage of memory (CD44⁺/CD11a⁺) T cells produced both IFNγ and IL17A when compared to naïve (CD44⁻/CD11a⁻) T cells. This was true 24 hrs. following both CLP and a sham procedure where the cecum is manipulated without either ligation or puncture, and, for IFNγ, of both lymphatic (splenic) and solid tissue resident (hepatic) T cells. Memory T cell IL17A production was only greater in the spleen in these experiments. This contrasts TNFα production, which is primarily produced by naïve T cells in the spleen. No difference in TNFα production was observed in the liver. Memory and naïve T cells produced IL2 at approximately the same frequency, but this response was significantly suppressed by CLP when compared to sham procedure. CD4 and CD8 T cells demonstrated similar responses overall, though CLP specifically augmented hepatic CD8 T cell IFNγ production, while it suppressed splenic CD4 IFNγ and IL17A production. Of note, the specificity of memory phenotype T cells in unchallenged mice maintained in specific pathogen free environments is unknown to date and the quality of the memory response by these cells is still controversial; these mice theoretically have not had pathogenic exposures to introduce antigenic stimulation to allow memory development.(17) Recent human studies have indicated that cross-reactivity to microbiomic or self-antigen may give rise to this population.(18, 19)

While CLP primarily had a partially suppressive effect on memory T cell effector responses when compared to sham procedure, we noted maintenance of differential memory versus naïve response even following challenge. When splenic memory and naïve T cells were examined for polyfunctional cytokine responses (i.e. production of >1 cytokine simultaneously), memory T cells continued to demonstrate a higher frequency of polyfunctional responses compared to naïve T cells. Both CLP and memory T cell status effected the number of cytokines produced in both CD4 and CD8 T cells. As memory T cells are able to maintain cytokine production effector responses following CLP, we set out to determine if induced-memory T cell cytokine production could alter immune responses and organ dysfunction in response to sepsis.

### T Cell Memory and Specific Cytokine Blockade Interact to Alter Serum Cytokine Responses to CLP

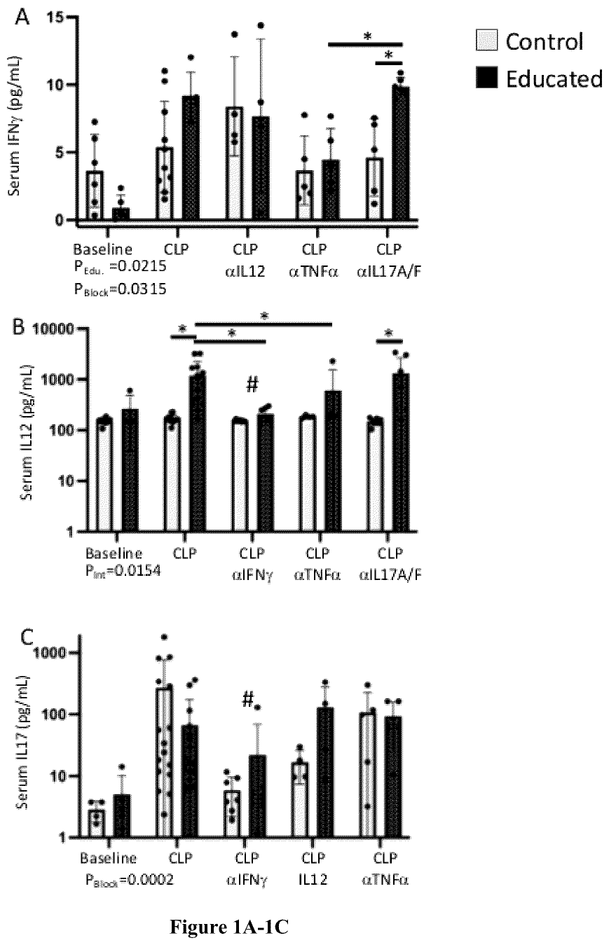

T cell memory responses to CLP cause higher levels of circulating IFNγ and IL12.(6) To delineate other effects of T cell memory responses on CLP, serum multiplex ELISA was performed on samples from control and Educated mice 24 hrs. following CLP (FIG. 1). Immune Education following in vivo administration of an activating anti-CD38 antibody induces wide-spread, non-specific T cell activation acutely with T cell memory development by 35 days and was used to study the effects of combined CD4 and CD8 T cell memory on the systemic response to CLP.(13) Subgroups of control and Educated mice subjected to CLP were treated with IFNγ, IL12, TNFα or IL17A/F blocking antibodies. Immune education led to higher levels of IFNγ in the serum 24 hrs. following CLP (FIG. 1A). Of note, the levels of IFNγ-inducible chemokines CXCL9 (MIG) and CXCL10 (IP10) were significantly higher than baseline in all animals subjected to CLP, indicating that CLP independently promotes IFNγ signaling, though Immune Education did not have effects on these chemokines, likely due to maximal effect. IL12 levels were similarly higher in mice subjected to CLP following Immune Education (FIG. 1B). IL12 has been shown to increase in response to IFNγ in other systems.(9, 20, 21) To examine if this effect was driving IL12 elevation in Educated CLP, we administered an IFNγ blocking antibody to control and Immune Educated mice at the time of CLP. The increase in serum IL12 induced by Immune Education in response to CLP was abrogated with IFNγ blockade (FIG. 1B). Blockade of IFNγ also led to suppression of elevations of serum IL17 induced by CLP independently of Immune Education (FIG. 1C). As previous studies have demonstrated interplay of IL17 and IFNγ responses during acute inflammation (22-27), blockade of IL17A/F (which are the primary IL17 family members produced by T cells) was pursued. Blockade of IL17A/F had no effect on Immune Education-induced IFNγ elevation following CLP, nor on IL12 elevation (FIG. 1A, 1B). In contrast, IFNγ blockade did suppress serum IL17 levels in both control and Educated mice following CLP (FIG. 1C).

As TNFα can be a T cell derived cytokine, we pursued blockade of TNFα in control and Educated CLP. No impact of TNFα blockade on serum IFNγ or IL17 could be detected (FIG. 1A, 1C), but TNFα blockade did suppress elevations in IL12 and IL1β in Educated CLP (FIG. 1B). Levels of CXCL10/IP10 were lower with TNFα blockade in Educated CLP and TNFα blockade led to lower levels of IP10 following CLP when compared to IL12 blockade. No changes with blockade were found in serum CXCL9/MIG levels.

In contrast to serum TNFα or IL13, blockade of IL17A/F decreased levels of IL6 independently of Immune Education, but had no effect on IL1β or TNFα, while TNFα blockade led to higher serum levels of IL6, again independently of Immune Education. IL10 levels, while unaffected by Immune Education, were lower with blockade of both IFNγ and IL17A/F, while serum IL10 was significantly higher with blockade of IL12. Blockade of TNFα had no effect on serum IL10. Finally, when chemokines were examined, MIP1β was higher with IL12 blockade and lower with IL17A/F blockade. Immune Education had no effect on this chemokine following CLP. Conversely MCP1 was lower with IFNγ blockade, but higher with IL17A/F blockade in Educated mice but not in control mice, possibly reflecting higher levels of IFNγ with IL17A/F blockade.

CD8 T Cells Contribute IL17F and IFNγ to Immune Educated CLP, while CD4 T Cells Contribute IL17A

Ex vivo T cell stimulation assays may be skewed by both the type of stimulation used and inherent indeterminable in vivo effects altering T cell responses. Therefore, use of transgenic reporter murine strains could provide significant detail concerning direct in vivo cytokine responses that could differ from ex vivo assays. Considering the significant influence of IFNγ on systemic serum cytokine responses in educated CLP, the direct in vivo source of the higher serum IFNγ levels observed in educated CLP was examined through comparison of the T cell and innate immune responses in Educated and control GREAT mice (IFNγ-eYFP reporter mice, B6.129S4-Ifng^{tm31Lky}/J) 24 hrs. following CLP. Education alone induced a higher percentage of IFNγ⁺ CD8 T cells in the spleen and a higher percentage of both IFNγ⁺ CD4 and IFNγ⁺ CD8 T cells in the liver prior to CLP. No change was found in splenic CD4 T cells. Following CLP, the percentage of hepatic IFNγ⁺ CD4 and CD8 T cells was reduced and was similar in both control and educated mice. In the spleen, a persistently higher percentage of CD8 T cells continued to produce IFNγ. The majority of IFNγ+ cells expressed a memory 10 phenotype: splenic IFNγ⁺ CD8 T cells were almost universally CD44^{hi}/CD62L^{lo}/CD11a⁺. These cells also expressed increased markers of activation, with an increased percentage of cells expressing CD69 and PD1 when compared to IFNγ T cells. In the liver, memory CD44⁺CD11a⁺ T cells were the primary IFNγ+ T cells and changes in the percentage of memory T cells corresponded to changes in percentage of the IFNγ+ memory T cell populations. Notably, no changes were detected in expression of IFNγ in the innate immune response or NK cell response to CLP in the presence of T cell memory induced by Education. Previous studies have identified IFNγ production by both myeloid cells—specifically a monocyte/macrophage population induced by CLP—and a small population of NK and NKT cells, though these are not influenced by Immune Education, indicating IFNγ induced changes in Educated CLP may be driven by IFNγ⁺ CD8 T cells.(28)

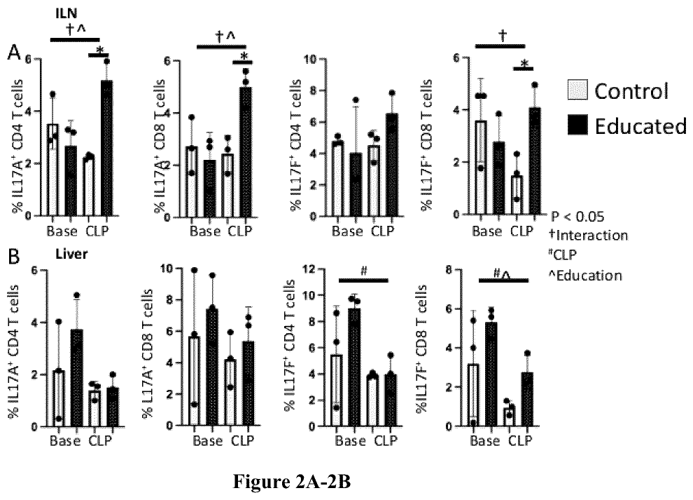

While memory CD8 T cells contribute IFNγ in promoting the immunophenotype observed with Educated CLP, memory CD4 T cells contributed to development of hepatic MoDCs and are apparently necessary to induce the full immune phenotype caused by Immune Education in CLP.(6) While total serum IL17 was increased by CLP, but not effected by Immune Education, at the cellular level, T cell-derived IL17A or IL17F could be altered by Immune Education in response to CLP, thereby altering microenvironmental signaling during T cell/Antigen presenting cell interactions. To assess the sources of IL17A and IL17F in Educated CLP, T cell and innate immune responses were examined 24 hrs. following CLP in control and Educated C57Bl/6-Il17a^{tm1Bcgen}Il17^{fem1Litt}/J IL17A^{EGFP}/IL17F^{mChery} reporter mice (FIG. 2A, 2B). Education alone had minimal effect on the percentage of IL17A or IL17F producing CD4 or CD8 T cells in the spleen prior to CLP. CLP induced a higher percentage of splenic IL17A⁺ CD4 T cells. In the inguinal lymph node (ILN), more proximal to the abdominal site of bacterial contamination in CLP, the proportion of CD4 and CD8 T cells producing IL17A was markedly higher following CLP in Educated mice when compared to control mice, an effect that was not apparent prior to challenge. In the liver, there were no changes noted in the percentage of CD4 or CD8 T cells producing IL17A, though there was a trend toward lower IL17A production in both lineages. In contrast to IL17A, IL17F production by CD4 T cells was unchanged by either education or CLP in the spleen or ILN and was depressed by CLP in the liver. In contrast to CD4 T cells, CD8 T cells demonstrated a preservation of IL17F production in the ILN following CLP, and, in the liver, IL17F production by CD8 T cells was increased by Immune Education both before and after CLP. IL17A⁺ T cells again generally expressed a memory phenotype, similar to IFNγ⁺ T cells: ILN IL17A⁺ CD4 T cells were primarily CD44^{hi}/CD62L^{lo}/CD11a⁺ following CLP. These T cells also demonstrated increased activation with an increased percentage of cells expressing CD69 and PD1, and proliferation as indicated by Ki67 expression when compared to IL17A-T cells. This phenotype is similar to that seen in IFNγ⁺ CD8 T cells from IFNγ-eYFP reporter mice. Surprisingly, T cells seemed to exclusively produce either IL17A or IL17F. Further, IL17F⁺ CD4 T cells did not display a memory phenotype and were largely CD44^{lo}/CD62L^{lo}/CD11a⁻, indicating an acute naïve effector T cell phenotype. These cells also did not have increases in CD69, PD1 or Ki67 expression. This is consistent with previous reports that IL17F is produced early in immune responses, while IL17A indicates a more committed Type 17 immune response.(29) IL17A⁺ CD4 and CD8 and IL17F⁺ CD4 and CD8 T cells had similar surface markers. Minimal Il17A⁺ or IL17F production was noted in innate immune cells.

### IL12 Suppresses T Cell TNFα Production Following CLP

Memory T cells produce a range of cytokine responses that can vary due to prior exposures or due to environmental factors at the time of response. This could lead skewing toward an IL17- or IFNγ-mediated immune response. Therefore, early exposure to IL17A or IFNγ in response to CLP—i.e. production by memory T cells as soon as CLP is performed or as soon as an infection begins—could significantly shift both T cell immune responses and broader immunity toward a Type 1 or Type 17 immune response. To examine the effects of this type of immune response skewing, PMA/Ionomycin stimulation was pursued ex vivo to assess effects of specific cytokine blockade on T cell cytokine production following CLP in control and Immune Educated mice, When CD4 T cells were assessed, IL12 blockade significantly enhanced the percentage of CD4 T cells producing IFNγ and IL17A in Educated CLP and TNFα in both control and Educated CLP, indicating that IL12 (which is increased in response to IFNγ) suppresses general CD4 T cell responses, though with differing effects in control and Immune Educated CLP. Blockade of IFNγ had minimal effects on IFNγ production by CD4 T cells and suppressed IL17A production, while blockade of IL17A/F in vivo suppressed IL17A production by CD4 T cells, indicating a feedforward IL17 response following CLP, consistent with IL17A promoting maturation of the Type 17 T cell immune response.(29) IL17A/F blockade also suppressed TNFα production in CD4 T cells. TNFα blockade depressed the percentage of CD4 T cells producing IL17A production following Educated CLP—this was not noted in control CLP. When hepatic CD8 T cell cytokine responses were assessed, the percentage of IFNγ⁺ CD8 T cells was enhanced by Immune Education, and TNFα blockade prevented this increase in IFNγ⁺ CD8 T cells. In contrast, blockade of IL17A/F significantly enhanced IL17A production by CD8 T cells in both Educated and control mice following CLP. CD8 T cell TNFα production was enhanced by blockade of IL12, again indicating that IL12, which is increased in response to memory T cell-derived IFNγ, may suppress T cell TNFα responses, which in turn facilitates T cell-derived IFNγ.

### IFNγ Suppresses Hepatic Neutrophil Infiltration and Promotes Hepatic MoDC Accumulation and IL12 Production Following CLP

T cell memory caused an increase in neutrophilic infiltration of the liver and more severe hepatic dysfunction following CLP.(6) IFNγ can promote neutrophil activation, which could cause hepatic dysfunction.(30, 31) IL17 similarly can augment a TNFα-driven response, which in turn promotes innate immunity.(32-34) To examine the connection between IFNγ and IL17 and enhancement of hepatic neutrophilic infiltration following CLP, IFNγ, TNFα, IL17A and IL17F were blocked at the time of CLP and the innate immune response in the liver was examined 24 hrs. following challenge. As IL12 suppressed T cell IFNγ and TNFα responses, and has been shown to promote IFNγ-promoting monocytic responses,(27) the role of IL12 in modification of innate immune responses was also assessed.

Both Immune Education and blockade of systemic IFNγ following CLP separately led to an enhanced hepatic neutrophilic response in Educated animals, with the most neutrophils noted in Educated animals that underwent IFNγ blockade. These data indicate that hepatic neutrophilic infiltration is induced by T cell memory independently of enhanced IFNγ production and that IFNγ may suppress hepatic neutrophil infiltration. In contrast to the hepatic neutrophil response, hepatic monocyte derived dendritic cells (MoDCs) in Educated CLP were suppressed by IFNγ blockade. This suppression did not occur in response to IFNγ-blocked CLP without the presence of pre-existing T cell memory, though hepatic MoDCs are not significantly higher without the presence of pre-existing memory. Hepatic inflammatory Ly6C^{hi}CD64⁺ marcrophages demonstrated a similar response with suppression of the number of these cells by IFNγ blockade in Educated CLP.

T cell memory induced both IL1β and TNFα production by inflammatory macrophages in response to CLP.(6) IFNγ blockade though did not have a detectable effect on neutrophil cytokine TNFα or IL1β production (data not shown). Hepatic MoDC and inflammatory macrophage cytokine production, in contrast, was significantly skewed by IFNγ blockade. Following CLP, in mice without pre-existing T cell memory, a large percentage of hepatic MoDCs and macrophages produced TNFα and IL1β following ex vivo LPS stimulation. IL12 production by these cell lines following CLP without prior induction of T cell memory was no different from baseline. TNFα production in these cell populations in standard CLP is unaffected by IFNγ blockade, while IFNγ blockade increased the percentage IL1β⁺ MoDCs following CLP with a statistical difference noted in Educated CLP with IFNγ blockade compared to mice without blockade. A similar increase in the percentage of MoDCs producing TNFα was also noted with IFNγ blockade only in Educated CLP. In Educated mice, a significantly higher percentage of hepatic MoDCs and macrophages produce IL12 following CLP, This is in response to IFNγ, as in vivo blockade of IFNγ suppresses the development of these activated IL12⁺ MoDC and macrophage populations.

### IL12 Reinforces IFNγ-Driven Hepatic MoDC Accumulation Following CLP

As IFNγ promoted IL12 production by hepatic MoDCs and macrophages, and as IL12 suppressed CD8 T cell-derived TNFα and CD4 T cell TNFα, IL17A and IFNγ, IL12⁺ MoDCs may act to reinforce the CD8 T cell IFNγ response. Similar effects have been observed through in vitro studies in human cells.(35) When IL12 was blocked in control and educated CLP, there were no effects on hepatic neutrophils or macrophage numbers. In contrast, IL12 blockade significantly reduced hepatic MoDCs in the livers of both control and Educated CLP mice. Importantly, no significant alterations were observed in innate immune cell cytokine production in the setting of IL12 blockade, though no differences between control and Educated mice could be detected due to high variability, which could be due to alterations by IL12 blockade. This included a lack of effect on the percentage of IL12⁺ MoDCs, indicating that IL12 production was dependent on IFNγ and that IL12 further reinforced the general MoDC response, as opposed to the reverse effect.

### TNFα Drives Increased Hepatic Neutrophilic and MoDC Responses in Educated CLP

TNFα is known to have wide ranging effects in both sepsis and CLP. We found that serum TNFα is higher in Educated CLP compared to control and facilitates both CD4 T cell IL17A production and CD8 T cell IFNγ production, indicating an essential role in licensing memory T cells to respond to CLP. As such, TNFα could facilitate innate immune responses to CLP that modulate memory T cell activation. Blockade of TNFα abrogated any difference between control and Educated CLP in hepatic neutrophil number, and suppressed hepatic MoDC and macrophage accumulation regardless of T cell memory status in CLP. TNFα blockade had no appreciable effects on TNFα or IL12 production by either MoDCs or macrophages, but did lead to a suppression of IL1β production by these lineages. The role of TNFα in promoting IL1β could indicate an essential role of CD4 T cell-derived TNFα, in promoting increased serum levels of IL1β in Educated CLP through interactions with MoDCs and macrophages.

IL17F is Necessary for MoDC Accumulation in the Liver, while IL17A Suppresses Hepatic Neutrophil Responses Following CLP

IL17 has previously been noted to augment effects of TNFα on neutrophil responses and can have broad effects on innate immunity.(32-34) Considering increased T cell IL17 production and serum levels in CLP, IL17 may promote IL12⁺ MoDC development, which in turn promotes IFNγ production by CD8 T cells. The primary isoforms of IL17 produced by T cells are IL17A and IL17F. Previous reports have shown that IL17F may promote Type 1 T cell responses mediated through IL12⁺ monocytes.(22, 25-27, 29) As such, blockade of these IL17 isoforms individually and together was pursued in control and Educated CLP and hepatic innate immune responses were assessed. Immune Education led to an increased number of neutrophils in the liver 24 hrs. following CLP; when IL17A was blocked, significantly more hepatic neutrophils were found following CLP than were noted without blockade. This was irrespective of Immune Education and was not observed with IL17F blockade, indicating that IL17A suppresses hepatic neutrophil numbers following CLP in a manner similar to that of IFNγ. Effects of double blockade of IL17A/F were similar and indistinguishable from blockade of IL17A by itself, while IL17F had little apparent effect on neutrophil numbers. We have previously demonstrated that hepatic MoDCs are promoted by memory CD4 T cells induced through Education (6); when this lineage was examined following blockade of IL17A/F, the increase in this cell line in Educated CLP was lost. When IL17A alone was blocked, Immune Education continued to drive a significant accumulation of hepatic MODCs—this effect was lost with IL17F blockade, indicating that IL17F is necessary to allow the increase in MoDCs driven by Immune Education. This was similar to effects of IFNγ blockade, possibly indicating an interdependence of IL17F, MoDCs and IFNγ. A similar pattern was seen in hepatic Ly6C^{Hi}CD64⁺ macrophages. When innate immune cell cytokine production was assessed in these experiments, the percentages of hepatic IL12⁺ MoDCs and IL12⁺ macrophages were lower in the setting of blockade of IL17F alone, which could indicate a commensurate response accompanying lower overall numbers of MoDCs. In contrast, hepatic macrophage IL12 production was promoted by IL17A blockade indicating differential effects. IL17F blockade led to a lower percentage of TNFα producing MoDCs and IL1β producing MoDCs and macrophages, though minimal effect was seen with combined IL17A/F blockade.

Together this set of data would indicate that IL17A suppresses hepatic neutrophil accumulation in CLP independently of IL17F, while accumulation of hepatic MoDCs and macrophages requires IL17F—possibly indicating IL17F promotes initiation of a cascade promoting CD8 memory T cell IFNγ production, but that IL17A, in turn, suppresses these same responses, possibly through T cell and innate immune cell TNFα.

### Blockade of IL17F and IFNγ Ameliorates Hepatic Dysfunction in Immune Educated CLP

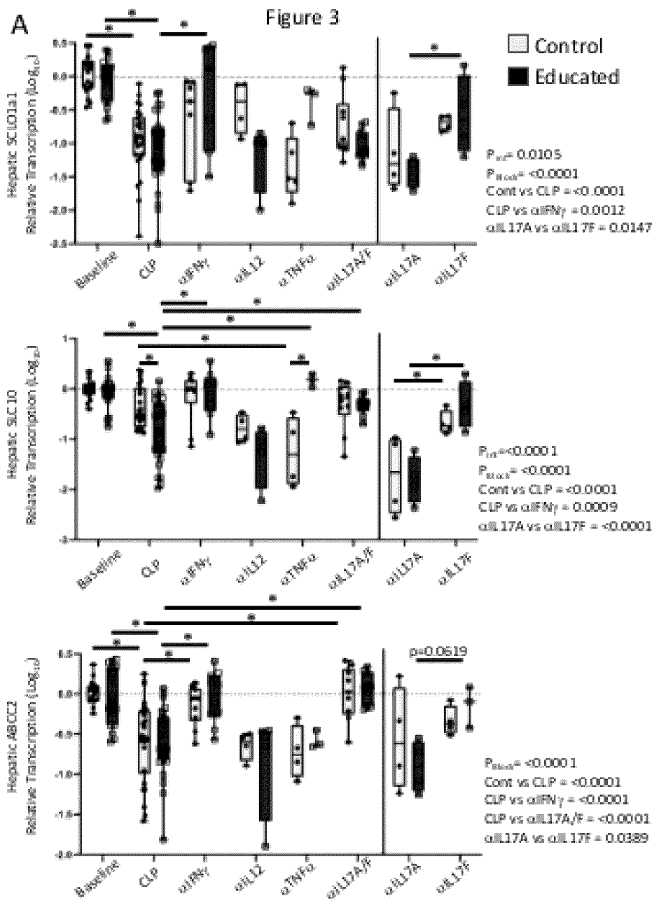

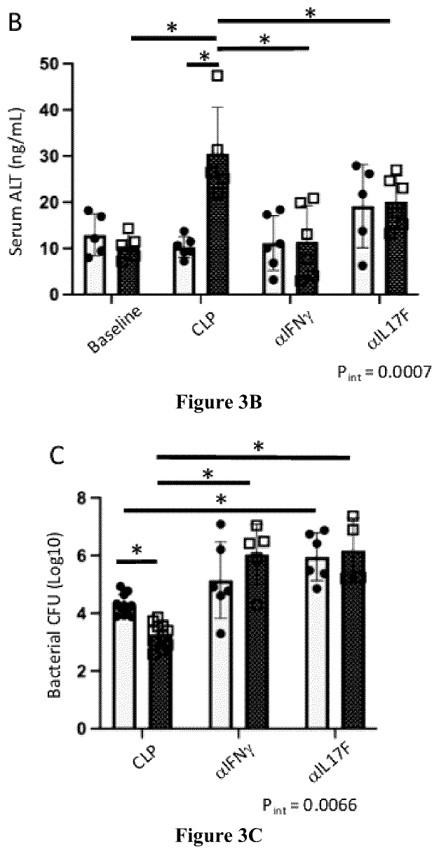

Previously, Immune Education was found to worsen hepatic dysfunction in response to CLP.(6) To assess if any of the cytokines studied herein affected transcription of bile acid transporters in the liver, control and Immune Educated mice were subjected to CLP with subgroups undergoing specific cytokine blockade at the time of CLP (FIG. 3A). Three separate transcripts, ABCC2, SLC10, and SCLO1a1, were assessed to increase the robustness of analysis of liver function—these have been previously demonstrated to be robust, sensitive markers of hepatic dysfunction in sepsis and all participate in hepatobiliary processes that are essential to normal function and are altered in sepsis-associated hepatic dysfunction.(36-38) All three transcripts were suppressed in Educated CLP, while two of the three, SCLO1a1 and ABCC2 were suppressed in control CLP. IFNγ blockade rescued transcription of all three of these bile acid transporters in Immune Educated mice. ABCC2 was rescued in control mice, while less of an effect of IFNγ was observed with SCLO1a1 transcription. IL12 had no effect on hepatic dysfunction. TNFα blockade led to improvement in SLC10 in educated animals following CLP, but no effect was noted in control CLP. ABCC2 was also rescued by dual blockade of IL17A and IL17F in both naïve and Educated mice, while SCL10 was rescued in Educated mice only, though it was not suppressed by CLP in control mice. Importantly, IL17A alone had minimal effect in improving transcription following CLP, while IL17F demonstrated improvement across the board, though not as robustly as IFNγ blockade.

Serum alanine aminotransferase (ALT) levels are utilized clinically for to identify patients with hepatic dysfunction in sepsis. To support findings of the amelioration of hepatic dysfunction with IFNγ and IL17F blockade identified through use of the transcriptional markers described in FIG. 3A, serum ALT levels were determined in control and Educated mice following CLP, or following CLP with blockade of IL17F and IFNγ. Serum ALT was significantly higher than baseline mice and control mice following CLP than in Educated mice following CLP. This rise in ALT was abrogated by blockade of IL17F and IFNγ (FIG. 3B).

### IL7F and IFNγ Contribute to Bacterial Clearance in Immune Educated CLP

While we have demonstrated that T cell memory responses contribute to hepatic dysfunction, morbidity and mortality in response to CLP, classically, memory T cells contribute significantly to enhanced clearance of bacterial infection and prevention of illness; a memory T cell response to most infections would lead to reduced symptomology, reduced time to bacterial clearance and improved outcomes. Indeed, when peritoneal bacterial count was assayed 24 hrs. following CLP in control and Immune Educated mice, T cell memory lead to reduction in bacterial colony forming units (CFUs), indicating enhanced immune responses. CLP is a model with persistent infection with tissue injury caused by ligation of the cecum with puncture to create a leaky bacterial pocket with reduced blood supply—essentially creating an abscess similar to a ruptured diverticulum or a perforated appendix. The ability of the immune response to eliminate the infecting bacteria in CLP is limited. Therefore, a more robust and persistent immune response guided by T cell memory in Educated CLP may contribute to organ dysfunction incidentally while attempting to eliminate peritoneal bacteria. To test this hypothesis, peritoneal bacterial counts were examined in control and Educated mice 24 hrs. following CLP with IL17F or IFNγ blockade (FIG. 3C). In both control and Educated animals blockade of IL17F led to higher bacterial CFUs compared to unblocked animals. Consistent with derivation of IFNγ from induced memory CD8 T cells, blockade of this cytokine led to higher bacterial CFUs only in Educated animals; this cytokine did not have a statistically detectable effect in control CLP.

### Discussion

Memory CD8 T cell-derived Interferon gamma promotes hepatic dysfunction in the cecal ligation and puncture model of sepsis. These effects depend on IL17F eliciting a monocyte derived dendritic cell response. Conversely, memory CD4 T cells produce TNFα and IL17A, to promote neutrophilic responses which do not affect hepatic function. These data demonstrate that a balance between the T cell mediated IL17A/IFNγ responses may determine development of hepatic dysfunction during the septic response.

Several important observations arise from these data. Perhaps the point of highest importance is the ability of the T cell cytokine response to modulate the systemic response to CLP. The human T cell immune compartment is primarily composed of memory T cells that will likely be the first responders to a systemic infection that could cause sepsis. An appropriately directed T cell response should prevent an infection from becoming systemic and causing sepsis; these data could indicate that a misdirected T cell response may allow sepsis to develop. Further, the ability of T cell-derived IFNγ to drive hepatic dysfunction may indicate that T cell activation causing cytokine production could contribute to organ dysfunction in general. Previous work in this area has shown that CD8 T cells can promote liver dysfunction in CLP(43). The mechanisms behind these findings are still unclear, though work in hemophagocytic lymphohistiocytosis has demonstrated that IFNγ can cause intrinsic hepatocyte injury directly through IFNγ receptor signaling.(12) The role of IL17F in allowing the CD8 T cell IFNγ response is also novel and requires further investigation—few studies have differentiated IL17F and IL17A in the past due to difficulty with reagents and model availability. Our results definitively show that these two isoforms of IL17 differentially mediate both immune responses and organ function in CLP. Further, IFNγ blockade may represent a viable treatment for sepsis patients with hepatic dysfunction. In addition, directed IL17F inhibition, as opposed to currently available combined IL17A/IL17F or directed IL17A inhibiting reagents, represents an immune modulating target in sepsis.

Previous studies have indicated that IL17F could drive IFNγ T cell responses. In the experimental colitis model of inflammatory bowel disease, IL17F⁺ CD4 T cells represent an early part of developmental pathogenesis and contribute to the differentiation of Th1 IFNγ⁺ T cells that ultimately drive continued disease.(25) Further studies have demonstrated plasticity of these IL17F T cells with the ability to go on to form IL17A⁺ Th17 cells or IFNγ+Th1 cells.(29) The balance between the Th17 and Th1 differentiation has been partially attributed to innate immune signaling facilitated by IL1β or IL18 to allow activation through IL12 or IL23, to firmly promote either IFNγ or 1117A T cell responses respectively.(44) It is intriguing to hypothesize that a similar balance may play out in human sepsis which could lead to varied clinical organ dysfunction or sepsis phenotypes.

Our findings also again indicate that T cell memory responses modulate innate immunity and organ function in CLP and sepsis. We and others have shown that T cells could contribute to hyperinflammatory responses to sepsis.(7, 45, 46) Our current data indicate that T cell responses causes worsened hepatic dysfunction, but there may be benefits to this increased inflammatory response that may include increased innate immune function and possibly suppression or increased clearance of infectious sources.

The ability of IFNγ in driving immune responses to clear infection has been previously established, though studies in its role in failed immunity with disseminated infection are more limited. Soudja et al. showed that memory T cell production of IFNγ is essential for control of Listeria monocytogenes infection, which is mediated through modulation of innate immune responses.(30) Minimal investigation into the role of IFNγ in CLP has been pursued but studies did demonstrate improvement in survival in IFNγ^{-/-} animals with antibiotic treatment, possibly indicating a role in promotion of both inflammatory responses and bacterial clearance in CLP.(28)

Indeed, while blockade of the IL17F and IFNγ may reduce hepatic dysfunction in our model, this cytokine axis also plays an apparent role in bacterial control, another vital component in stopping sepsis. The finding that T cell memory contributes positively to boost immune driven clearance of bacteria is not novel as adaptive immunity is widely protective against a broad range of pathogens. Contrary to this, our work would indicate that, while T cell memory does control bacterial load in CLP, it also contributes to significant morbidity. CLP is a model with high levels of persistent infection—infection that the immune response tries to clear without success. The implications of these findings are profound; sepsis is caused by a dysregulated response to infection—an infection with a pathogenic burden that is causing harm and that the body has already failed to clear. If the immune system does not curb its response, off-target inflammation as seen in our model driven by T cell memory, causes morbidity and mortality. This finding brings to question whether the sepsis-associated immunosuppression in T cells, characterized by widespread apoptosis and T regulatory cell development, is truly maladaptive, or whether it may serve a vital purpose in allowing survival when the immune response cause more harm than the infection itself.

One of the salient aspects inherent in these findings is that, as cytokine responses by T cells are inherently an activation event, T cells are undergoing activation in response to CLP. Investigations into the factors promoting of this activation have yielded limited definitive answers beyond apparently non-specific “cytokine-driven” activation.(47) These experiments show the importance of understanding the nature of this activation, along with possibly the importance of the directionality of the activation—different T cell clones may produce differential cytokine responses, which may lead to varying outcomes in both CLP and sepsis. Similarly, the variation in T cell response may occur secondary to a myriad of variables present at the time of challenge. These include other cytokines, the activities of innate immune cells and antigen presenting cells, and the co-ligands present on these cells.

In summary, T cell memory responses significantly alter the immune response and organ dysfunction that occurs in response to the cecal ligation and puncture (CLP) model of sepsis, promoting both hepatic neutrophilic responses and causing hepatic dysfunction, which is a common complication of sepsis and associated with particularly poor outcomes. We used the Immune Educated CLP model to examine the role of memory T cell cytokine responses in driving innate immune and organ dysfunction in CLP. T cell Education was induced in mice 35 days prior to CLP through administration of the 145-2C11 anti-CD38 antibody, which leads to widespread CD4 and CD8 T cell memory induction. Induced T cell memory prior to CLP led to higher serum levels of IL12, TNFα, IL17, IL1β and IFNγ. Elevations in IL12, TNFα, IL17, and IL1β were all abrogated by IFNγ blockade. Use of IFNγ reporter mice demonstrated increased IFNγ production by memory. CD8 T cells in the liver prior to CLP and in the spleen both before and after CLP. IFNγ caused induction of hepatic IL12⁺ monocyte derived dendritic cells, which depended on IFNγ, TNFα and IL17F. Increased neutrophilic responses in Educated CLP conversely depended on TNFα, while IFNγ significant suppressed hepatic neutrophilic numbers, but led to increased functional responses with higher percentages producing TNFα and IL1β in the presence of IFNγ⁺ CD8 T cells. Hepatic dysfunction in response to CLP was worsened by Immune Memory and prevented by IFNγ and IL17F. Immune Memory induces an IL17F/IFNγ CD8 T cell response that reduces hepatic neutrophilic responses but causes worsened hepatic dysfunction in CLP. IL17F or IFNγ blockade represents a potential target for treatment in sepsis with hepatobiliary dysfunction.

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

### CITED PUBLICATIONS

1. Singer M, Deutschman C S, Seymour C W, Shankar-Hari M, Annane D, Bauer M, et al. The Third International Consensus Definitions for Sepsis and Septic Shock (Sepsis-3). JAMA. 2016; 315(8):801-10.
2. Osuchowski M F, Ayala A, Bahrami S, Bauer M, Boros M, Cavaillon J M, et al. Minimum quality threshold in pre-clinical sepsis studies (MQTiPSS): an international expert consensus initiative for improvement of animal modeling in sepsis. Intensive Care Med Exp. 2018; 6(1):26.
3. Beura L K, Hamilton S E, Bi K, Schenkel J M, Odumade O A, Casey K A, et al. Normalizing the environment recapitulates adult human immune traits in laboratory mice. Nature. 2016; 532(7600):512-6.
4. Abolins S, King E C, Lazarou L, Weldon L, Hughes L, Drescher P, et al. The comparative immunology of wild and laboratory mice, Mus musculus domesticus. Nat Commun. 2017; 8:14811.
5. Taylor M D, Brewer M R, Nedeljkovic-Kurepa A, Yang Y, Reddy K S, Abraham M N, et al. CD4 T Follicular Helper Cells Prevent Depletion of Follicular B Cells in Response to Cecal Ligation and Puncture. Front Immunol. 2020; 11:1946.
6. Taylor M D, Fernandes T D, Kelly A P, Abraham M N, Deutschman C S. CD4 and CD8 T Cell Memory Interactions Alter Innate Immunity and Organ Injury in the CLP Sepsis Model. Front Immunol. 2020; 11:563402.
7. Huggins M A, Sjaastad F V, Pierson M, Kucaba T A, Swanson W, Staley C, et al. Microbial Exposure Enhances Immunity to Pathogens Recognized by TLR2 but Increases Susceptibility to Cytokine Storm through TLR4 Sensitization. Cell Rep. 2019; 28(7):1729-43 e5.
8. Rudd K E, Johnson S C, Agesa K M, Shackelford K A, Tsoi D, Kievlan D R, et al. Global, regional, and national sepsis incidence and mortality, 1990-2017: analysis for the Global Burden of Disease Study. Lancet. 2020; 395(10219):200-11.
9. Taylor M D, Fernandes T D, Yaipen O, Higgins C E, Capone C A, Leisman D E, et al. T cell activation and IFNgamma modulate organ dysfunction in LPS-mediated inflammation. J Leukoc Biol. 2022.
10. Anderko R R, Gomez H, Canna S W, Shakoory B, Angus D C, Yealy D M, et al. Sepsis with liver dysfunction and coagulopathy predicts an inflammatory pattern of macrophage activation. Intensive Care Med Exp. 2022; 10(1):6.
11. Kobashi H, Toshimori J, Yamamoto K. Sepsis-associated liver injury: Incidence, classification and the clinical significance. Hepatol Res. 2013; 43(3):255-66.
12. Diamond T, Burn T N, Nishiguchi M A, Minichino D, Chase J, Chu N, et al. Familial hemophagocytic lymphohistiocytosis hepatitis is mediated by IFN-gamma in a predominantly hepatic-intrinsic manner. PLoS One. 2022; 17(6):e0269553.
13. Taylor M D, Brewer M R, Deutschman C S. Induction of diverse T cell memory through antibody-mediated activation in mice. Eur J Immunol. 2020; 50(11):1838-40.
14. Jameson S C, Masopust D. Understanding Subset Diversity in T Cell Memory. Immunity. 2018; 48(2):214-26.
15. Sallusto F, Lanzavecchia A, Araki K, Ahmed R. From vaccines to memory and back. Immunity. 2010; 33(4):451-63.
16. Williams M A, Bevan M J. Effector and memory CTL differentiation. Annu Rev Immunol. 2007; 25:171-92.
17. Lee J Y, Hamilton S E, Akue A D, Hogquist K A, Jameson S C. Virtual memory CD8 T cells display unique functional properties. Proc Natl Acad Sci USA. 2013; 110(33):13498-503.
18. Goncalves P, El Daker S, Vasseur F, Serafini N, Lim A, Azogui O, et al. Microbiota stimulation generates LCMV-specific memory CD8(+) T cells in SPF mice and determines their TCR repertoire during LCMV infection. Mol Immunol. 2020; 124:125-41.
19. Bartolo L, Afroz S, Pan Y G, Xu R, Williams L, Lin C F, et al. SARS-CoV-2-specific T cells in unexposed adults display broad trafficking potential and cross-react with commensal antigens. Sci Immunol. 2022; 7(76):eabn3127.
20. Goplen N P, Saxena V, Knudson K M, Schrum A G, Gil D, Daniels M A, et al. IL-12 Signals through the TCR To Support CD8 Innate Immune Responses. J Immunol. 2016; 197(6):2434-43.
21. Rottinghaus E K, Vesosky B, Turner J. Interleukin-12 is sufficient to promote antigen-independent interferon-gamma production by CD8 T cells in old mice. Immunology. 2009; 128(1 Suppl):e679-90.
22. Hou L, Jie Z, Desai M, Liang Y, Soong L, Wang T, et al. Early IL-17 production by intrahepatic T cells is important for adaptive immune responses in viral hepatitis. J Immunol. 2013; 190(2):621-9.
23. Kim O Y, Hong B S, Park K S, Yoon Y J, Choi S J, Lee W H, et al. Immunization with Escherichia coli outer membrane vesicles protects bacteria-induced lethality via Th1 and Th17 cell responses. J Immunol. 2013; 190(8):4092-102.
24. Bagri P, Anipindi V C, Nguyen P V, Vitali D, Stampfli M R, Kaushic C. Novel Role for Interleukin-17 in Enhancing Type 1 Helper T Cell Immunity in the Female Genital Tract following Mucosal Herpes Simplex Virus 2 Vaccination. J Virol. 2017; 91(23).
25. Harbour S N, Maynard C L, Zindl C L, Schoeb T R, Weaver C T. Th17 cells give rise to Th1 cells that are required for the pathogenesis of colitis. Proc Natl Acad Sci USA. 2015; 112(22):7061-6.
26. Feng T, Qin H, Wang L, Benveniste E N, Elson C O, Cong Y. Th17 cells induce colitis and promote Th1 cell responses through IL-17 induction of innate IL-12 and IL-23 production. J Immunol. 2011; 186(11):6313-8.
27. Davidson M G, Alonso M N, Yuan R, Axtell R C, Kenkel J A, Suhoski M M, et al. Th17 cells induce Th1-polarizing monocyte-derived dendritic cells. J Immunol. 2013; 191(3):1175-87.
28. Romero C R, Herzig D S, Etogo A, Nunez J, Mahmoudizad R, Fang G, et al. The role of interferon-gamma in the pathogenesis of acute intra-abdominal sepsis. J Leukoc Biol. 2010; 88(4):725-35.
29. Lee Y K, Turner H, Maynard C L, Oliver J R, Chen D, Elson C O, et al. Late developmental plasticity in the T helper 17 lineage. Immunity. 2009; 30(1):92-107.
30. Soudja S M, Chandrabos C, Yakob E, Veenstra M, Palliser D, Lauvau G. Memory-T-cell-derived interferon-gamma instructs potent innate cell activation for protective immunity. Immunity. 2014; 40(6):974-88.
31. Ellis T N, Beaman B L. Interferon-gamma activation of polymorphonuclear neutrophil function. Immunology. 2004; 112(1):2-12.
32. Beringer A, Thiam N, Molle J, Bartosch B, Miossec P. Synergistic effect of interleukin-17 and tumour necrosis factor-alpha on inflammatory response in hepatocytes through interleukin-6-dependent and independent pathways. Clin Exp Immunol. 2018; 193(2):221-33.
33. Griffin G K, Newton G, Tarrio M L, Bu D X, Maganto-Garcia E, Azcutia V, et al. IL-17 and TNF-alpha sustain neutrophil recruitment during inflammation through synergistic effects on endothelial activation. J Immunol. 2012; 188(12):6287-99.
34. Noack M, Beringer A, Miossec P. Additive or Synergistic Interactions Between IL-17A or IL-17F and TNF or IL-1beta Depend on the Cell Type. Front Immunol. 2019; 10:1726.
35. Miro F, Nobile C, Blanchard N, Lind M, Filipe-Santos O, Fieschi C, et al. T cell-dependent activation of dendritic cells requires IL-12 and IFN-gamma signaling in T cells. J Immunol. 2006; 177(6):3625-34.
36. Abraham M N, Kelly A P, Brandwein A B, Fernandes T D, Leisman D E, Taylor M D, et al. Use of Organ Dysfunction as a Primary Outcome Variable Following Cecal Ligation and Puncture: Recommendations for Future Studies. Shock. 2020; 54(2):168-82.
37. Andrejko K M, Raj N R, Kim P K, Cereda M, Deutschman C S. IL-6 modulates sepsis-induced decreases in transcription of hepatic organic anion and bile acid transporters. Shock. 2008; 29(4):490-6.
38. Kim P K, Chen J, Andrejko K M, Deutschman C S. Intraabdominal sepsis down-regulates transcription of sodium taurocholate cotransporter and multidrug resistance-associated protein in rats. Shock. 2000; 14(2):176-81.
39. Matics T J, Sanchez-Pinto L N. Adaptation and Validation of a Pediatric Sequential Organ Failure Assessment Score and Evaluation of the Sepsis-3 Definitions in Critically Ill Children. JAMA Pediatr. 2017; 171(10):e172352.
40. Menten P, Proost P, Struyf S, Van Coillie E, Put W, Lenaerts J-P, et al. Differential induction of monocyte chemotactic protein-3 in mononuclear leukocytes and fibroblasts by interferon-α/β and interferon-7 reveals MCP-3 heterogeneity. European Journal of Immunology. 1999; 29(2):678-85.
41. Ge M Q, Ho A W, Tang Y, Wong K H, Chua B Y, Gasser S, et al. N K cells regulate CD8+ T cell priming and dendritic cell migration during influenza A infection by IFN-gamma and perforin-dependent mechanisms. J Immunol. 2012; 189(5):2099-109.
42. Hurgin V, Novick D, Werman A, Dinarello C A, Rubinstein M. Antiviral and immunoregulatory activities of IFN-gamma depend on constitutively expressed I L-Ialpha.
Proc Natl Acad Sci USA. 2007; 104(12):5044-9.
43. Wesche-Soldato D E, Chung C S, Gregory S H, Salazar-Mather T P, Ayala C A, Ayala A. CD8+ T cells promote inflammation and apoptosis in the liver after sepsis: role of Fas-FasL. Am J Pathol. 2007; 171(1):87-96.
44. Lee Y K, Landuyt A E, Lobionda S, Sittipo P, Zhao Q, Maynard C L. TCR-independent functions of Th17 cells mediated by the synergistic actions of cytokines of the IL-12 and IL-1 families. PLoS One. 2017; 12(10):e0186351.
45. Schmoeckel K, Traffehn S, Eger C, Potschke C, Broker B M. Full activation of CD4+ T cells early during sepsis requires specific antigen. Shock. 2015; 43(2):192-200.
46. Sherwood E R, Enoh V T, Murphey E D, Lin C Y. Mice depleted of CD8+ T and N K cells are resistant to injury caused by cecal ligation and puncture. Lab Invest. 2004; 84(12):1655-65.
47. Unsinger J, Herndon J M, Davis C G, Muenzer J T, Hotchkiss R S, Ferguson T A. The role of TCR engagement and activation-induced cell death in sepsis-induced T cell apoptosis. J Immunol. 2006; 177(11):7968-73.
48. Abcejo A S, Andrejko K M, Raj N R, Deutschman C S. Failed interleukin-6 signal transduction in murine sepsis: attenuation of hepatic glycoprotein 130 phosphorylation. Crit Care Med. 2009; 37(5):1729-34.
49. Abraham M N, Jimenez D M, Fernandes T D, Deutschman C S. Cecal Ligation and Puncture Alters Glucocorticoid Receptor Expression. Crit Care Med. 2018; 46(8):e797-e804.
50. Jordan M B, Hildeman D, Kappler J, Marrack P. An animal model of hemophagocytic lymphohistiocytosis (HLH): CD8⁺ T cells and interferon gamma are essential for the disorder. Blood. 2004; 104(3):735-43.
51. Taylor M D, Burn T N, Wherry E J, Behrens E M. CD8 T Cell Memory Increases Immunopathology in the Perforin-Deficient Model of Hemophagocytic Lymphohistiocytosis Secondary to TNF-alpha. Immunohorizons. 2018; 2(2):67-73.
52. Naik S, Bouladoux N, Linehan J L, Han S J, Harrison O J, Wilhelm C, et al. Commensal-dendritic-cell interaction specifies a unique protective skin immune signature. Nature. 2015; 520(7545):104-8.
53. Lemaire M M, Dumoutier L, Warnier G, Uyttenhove C, Van Snick J, de Heusch M, et al. Dual TCR expression biases lung inflammation in DO11.10 transgenic mice and promotes neutrophilia via microbiota-induced Th17 differentiation. J Immunol. 2011; 187(7):3530-7.
54. Sercan 0, Stoycheva D, Hammerling G J, Arnold B, Schuler T. IFN-gamma receptor signaling regulates memory CD8+ T cell differentiation. J Immunol. 2010; 184(6):2855-62.
55. Gerhardt S, Abbott W M, Hargreaves D, Pauptit R A, Davies R A, Needham M R, Langham C, Barker W, Aziz A, Snow M J, Dawson S, Welsh F, Wilkinson T, Vaugan T, Beste G, Bishop S, Popovic B, Rees G, Sleeman M, Tuske S J, Coales S J, Hamuro Y, Russell C. Structure of IL-17A in complex with a potent, fully human neutralizing antibody. J Mol Biol. 2009; 394(5):905-21. Epub 2009/10/20. doi: 10.1016/j.jmb.2009.10.008. PubMed PMID: 19835883.
56. Hymowitz S G, Filvaroff E H, Yin J P, Lee J, Cai L, Risser P, Maruoka M, Mao W, Foster J, Kelley R F, Pan G, Gurney A L, de Vos A M, Starovasnik M A. IL-17s adopt a cystine knot fold: structure and activity of a novel cytokine, IL-17F, and implications for receptor binding. EMBO J. 2001; 20(19):5332-41. Epub 2001/09/28. doi: 10.1093/emboj/20.19.5332. PubMed PMID: 11574464; PMCID: PMC125646.
57. Harbour S N, Maynard C L, Zindl C L, Schoeb T R, Weaver C T. Th17 cells give rise to Th1 cells that are required for the pathogenesis of colitis. Proc Natl Acad Sci USA. 2015; 112(22):7061-6. Epub 2015/06/04. doi: 10.1073/pnas.1415675112. PubMed PMID: 26038559; PMCID: PMC4460486.
58. Noack M, Beringer A, Miossec P. Additive or Synergistic Interactions Between IL-17A or IL-17F and TNF or IL-1beta Depend on the Cell Type. Front Immunol. 2019; 10:1726. Epub 2019/08/10. doi: 10.3389/fimmu.2019.01726. PubMed PMID: 31396230; PMCID: PMC6664074. ADDIN E N.REFLIST

What is claimed is:

1. A method of treating an infection or an organ dysfunction in a patient with a disease or disorder comprising modulating the level or activity of interleukin (IL)17F in the patient.

2. The method of claim 1, wherein the patient has an infection and supplementation of IL17F augments clearance of the infection in the patient.

3. The method of claim 2, wherein the infection is a bacterial, mycobacterial, fungal or viral infection.

4. The method of claim 2, wherein the infection is due to influenza, Epstein-Barr virus (EBV), Cytomegalovirus (CMV), a hemorrhagic fever virus or COVID19.

5. The method of claim 2, wherein the infection is a persistent chronic infection or a severe acute infection.

6. The method of claim 2, wherein the patient has sepsis and supplementation of IL17F augments bacterial clearance in the patient.

7. The method of claim 2, wherein the patient has pneumonia or hepatitis or encephalitis.

8. The method of claim 1, wherein blockade of IL17F ameliorates organ dysfunction in the patient.

9. The method of claim 8, wherein the organ dysfunction is hepatic, renal or lung dysfunction.

10. The method of claim 8, wherein the patient has sepsis and blockade of IL17F ameliorates hepatic dysfunction.

11. The method of claim 8, wherein the patient has severe trauma, a severe burn, brain injury/stroke, lung injury/acute respiratory distress syndrome (ARDS), drug overdose, acute renal failure (ARF), idiopathic liver failure, virally induced liver failure, hemophagocytic lymphohistiocytosis (HLH), macrophage activation syndrome (MAS), systemic Juvenile Idiopathic Arthritis (sJIA), systemic lupus erythematosus (SLE) or Kawasaki's disease (KD).

12. The method of claim 1, wherein the method does not comprise administering a substance to the patient that directly modulates the level or activity of IL17A.

13. The method of claim 1, wherein the patient is a human.

14. The method of claim 1, wherein the patient is a veterinary animal.
